(19) United States
(12) Patent Application Publication
(10) Pub. No.: US 2022/0370513 A1
(43) Pub. Date: Nov. 24, 2022

(54) COMPOSITION, ANTIOXIDANT AGENT, ANTISACCHARIFICATION AGENT, NEURITE OUTGROWTH PROMOTER, AND COGNITIVE FUNCTION IMPROVER

(71) Applicant: YAMADA BEE COMPANY, INC., Tomata-gun, Okayama (JP)

(72) Inventors: Tomomi ISHIKAWA, Tomata-gun, Okayama (JP); Nobuaki OKUMURA, Tomata-gun, Okayama (JP); Chika NISHIMORI, Tomata-gun, Okayama (JP); Takahiro FUJIOKA, Tomata-gun, Okayama (JP); Ayanori YAMAKI, Tomata-gun, Okayama (JP); Hiroko NAGAI, Tomata-gun, Okayama (JP); Takashi ASAMA, Tomata-gun, Okayama (JP)

(21) Appl. No.: 17/770,833

(22) PCT Filed: Oct. 28, 2020
(86) PCT No.: PCT/JP2020/040502

§ 371 Date: Apr. 21, 2022

(30) Foreign Application Priority Data
Nov. 1, 2019 (JP) 2019-200066
Jul. 17, 2020 (JP) 2020-122874

(51) Int. Cl. A61K 35/644; A61K 36/16; A61K 31/685; A61K 36/74; A61K 36/23; A61K 31/12; A61P 25/28

(52) U.S. Cl. CPC A61K 35/644; A61K 36/16; A61K 31/685; A61K 36/74; A61K 36/23; A61K 31/12; A61P 25/28

(57) ABSTRACT

There are disclosed an antioxidant agent, an antisaccharification agent, a neurite outgrowth promoting agent, and a cognitive function improving agent, which contain propolis and a ginkgo leaf extract as active ingredients.

### TECHNICAL FIELD

The present invention relates to a composition, an antioxidant agent, an antisaccharification agent, a neurite outgrowth promoting agent and a cognitive function improving agent.

### BACKGROUND ART

In recent years, the number of dementia patients has increased, which has become a social problem. In addition, many people, even non-dementia patients, suffer from forgetfulness or cognitive function decline. Known dementia disease types include Alzheimer's type dementia, vascular dementia, dementia with Lewy bodies, frontotemporal lobar degeneration (FTLD) and the like. The majority of dementia patients have Alzheimer type's dementia. Dementia involves accumulation of amyloid β proteins in the brain. For example, Patent Literature 1 discloses an oral composition containing a specific peptide as an active ingredient for improving brain dysfunctions caused by amyloid β.

### CITATION LIST

### Patent Literature

[Patent Literature 1] Japanese Unexamined Patent Publication No. 2018-154640

### SUMMARY OF INVENTION

### Technical Problem

Although many treatment methods for dementia have been investigated so far, these are only therapeutic drugs that temporarily delay the progress, and nothing that can be expected to have a fundamental treatment effect has been established. In particular, no fundamental treatment method has been established as a method for removing amyloid β. Prevention of cognitive function decline is important at a stage where symptoms are mild and in a reversible phase.

An object of the present invention is to provide a novel agent effective for improving cognitive functions.

### Solution to Problem

One aspect of the present invention relates to an antioxidant agent or an antisaccharification agent containing propolis and a ginkgo leaf extract as active ingredients.

The present invention also relates to a neurite outgrowth promoting agent containing propolis and a ginkgo leaf extract as active ingredients.

The present invention also relates to a cognitive function improving agent containing propolis and a ginkgo leaf extract as active ingredients.

The agent preferably does contain at least one of DHA and EPA.

Another aspect of the present invention relates to an antioxidant agent containing propolis, and at least one selected from the group consisting of phosphatidylserine, a coffee fruit extract and a gotu kola extract.

The present invention also relates to an antisaccharification agent containing propolis, and at least one selected from the group consisting of phosphatidylserine, a coffee fruit extract and curcumine.

The present invention also relates to a cognitive function improving agent containing propolis, and at least one selected from the group consisting of phosphatidylserine, a coffee fruit extract, a gotu kola extract and curcumine.

The present invention also relates to an anti-inflammatory agent containing propolis and curcumine.

Still another aspect of the present invention relates to a composition containing propolis, and at least one of phosphatidylserine and a coffee fruit extract.

The present invention relates to a foodstuff or a drug containing propolis and a gotu kola extract.

Yet another aspect of the present invention relates to a composition containing propolis, a ginkgo leaf extract, a coffee fruit extract, a gotu kola extract, curcumine and phosphatidylserine.

### Advantageous Effects of Invention

According to the present invention, there is provided a novel agent effective for improving cognitive functions.

### BRIEF DESCRIPTION OF DRAWINGS

FIG. 1 is a graph showing a neurite formation rate in Test Example 1.

FIG. 2 shows phase contrast microscopic images of PC12 cells in Test Example 1.

FIG. 3 is a graph showing an oxidative stress level in Test Example 2.

### DESCRIPTION OF EMBODIMENTS

Exemplary embodiments of the present invention will be described in detail below. However, the present invention is not limited to the following embodiments.

One aspect of the present embodiment relates to a composition containing propolis and at least one selected from the group consisting of a ginkgo leaf extract, phosphatidylserine, a coffee fruit extract, a gotu kola extract and curcumine. The composition may be a foodstuff, a quasi-drug or a drug.

The composition containing propolis and at least one selected from the group consisting of a ginkgo leaf extract, phosphatidylserine, a coffee fruit extract, a gotu kola extract and curcumine can be used as an antioxidant agent, an antisaccharification agent, a neurite outgrowth promoting agent and a cognitive function improving agent. If a ginkgo leaf extract, phosphatidylserine, a coffee fruit extract, a gotu kola extract and curcumine are taken in combination with propolis, compared to when these components are used alone, they exhibit strong effects in at least one action selected from the group consisting of antioxidation, antisaccharification and neurite outgrowth promoting actions, and have a high cognitive function improving effect based on these actions.

When propolis and curcumine are taken in combination, compared to when these components are used alone, a synergistic effect is exhibited for an Nrf2 pathway activation effect. It is known that Nrf2 pathway activation results in anti-inflammatory, antioxidation and cognitive function improving effects, and therefore, the composition containing propolis and curcumine can also be used as an anti-inflammatory agent in addition to the antioxidant agent or the cognitive function improving agent. In addition, the composition containing propolis and curcumine has liver function improving, sleep quality improving and cognitive function improving effects based on an anti-inflammatory effect. Therefore, the composition containing propolis and curcumine can also be used as a liver function improving agent or a sleep quality improving agent.

Another aspect of the present embodiment provides a composition containing propolis, a ginkgo leaf extract, phosphatidylserine, a coffee fruit extract, a gotu kola extract and curcumine. When the composition contains all of these components, it has a strong antioxidation effect, antisaccharification effect, anti-inflammatory effect and neurite outgrowth promoting effect, and has a strong sleep quality improving effect, liver function improving effect and cognitive function improving effect based on these effects. Hereinafter, respective components will be described.

(Propolis)

The propolis can be obtained as, for example, a beekeeping product according to a conventional method. Propolis itself has antioxidation, anti-inflammatory and cognitive function decline inhibition effects. The propolis may be derived from any plant such as alecrim-derived, eucalyptus-derived, poplar-derived and Clusia plant-derived propolis. In consideration of high physiological activity, alecrim-derived propolis is preferable. Alecrim is Baccharis dracunculifolia, which is the genus Baccharis belonging to the family Asteraceae.

The propolis may be produced in, for example, Japan, Brazil, China, European countries, Oceania and America. Brazilian propolis is mainly derived from alecrim. Brazilian propolis has a high content of cinnamic acid derivatives. The antioxidant agent, the antisaccharification agent, the neurite outgrowth promoting agent, the cognitive function improving agent, the anti-inflammatory agent, the liver function improving agent, and the sleep quality improving agent according to the present embodiment (hereinafter collectively referred to as an “agent according to the present embodiment”) preferably contain Brazilian propolis as an active ingredient.

The propolis may be any rank such as brown, red, yellow, green, super green and ultra green propolis, and among these, green, super green or ultra green rank propolis is preferable. These ranks are determined according to the content of artepillin C in propolis. Those containing 3 mass % or more of artepillin C are called green propolis. The content of artepillin C in the propolis is preferably 5 mass % or more and more preferably 8 mass % or more.

The propolis is preferably derived from a bee belonging to the genus Apis, family Apidae, and is preferably derived from the bee A. mellifera among bees of the genus Apis. There are 24 to 28 subspecies of A. mellifera, and propolis derived from any of the subspecies may be used. In particular, propolis derived from African honey bees, which are a hybrid of Africanized honey bees (A. mellifera scutellata), which are one subspecies of A. mellifera and other European subspecies of A. mellifera, is preferably used.

The propolis may be, for example, a propolis original lump or a processed propolis product obtained by performing some treatments on the propolis original lump. The processed propolis product may be, for example, a product obtained by performing treatments such as pulverization, extraction, concentration or powderizing of an extract, and powder granulation on the propolis original lump, or may be an extract residue remaining after extraction. That is, the processed propolis product may be, for example, a ground product of propolis, an extract, a concentrated extract, an extract powder, an extract granule, an extract residue or the like. Examples of extracts include a water extract, a hydrophilic organic solvent extract and a supercritical extract. Examples of hydrophilic organic solvents include ethanol, glycerin and 1,3-butylene glycol. The propolis extract may be a product obtained by extraction from the propolis original lump, or may be a product obtained by performing additional extraction from the residue after extraction. The treatment methods may be used alone or two or more thereof may be used in combination. Among the processed propolis products, the propolis-hydrophilic organic solvent extract is preferable because the active ingredient of propolis is efficiently extracted in a well-balanced manner in a short time. The processed propolis product is preferably a propolis ethanol extract.

As the propolis, commercial products may be used. Examples of commercial products containing propolis include Propolis 300, Propolis Liquid 30 (made in Brazil), Propolis Grains, Propolis Granule APC, Propolis Mild, Propolis Drink (commercially available from Yamada Bee Company, Inc.), Neopropolis Grains, Propolis Grains, Propolis Liquid, Propolis Mild Liquid, Eucalyptus Polis (commercially available from Morikawa Kenkodo Kk), L'abeille Propolis (liquid type), L'abeille Propolis (capsule type) and L'abeille Propolis Honey (commercially available from L'abeille).

The agent or composition can be used as a solid content of propolis, for example, at a dose of 1 mg to 1,000 mg, preferably at a dose of 10 to 500 mg, and more preferably at a dose of 20 to 200 mg per day for an adult with a body weight of 60 kg.

In addition, the content of propolis in the agent or composition may be, for example, 1 mass % or more, 3 mass % or more, 4 mass % or more or 5 mass % or more, and may be 30 mass % or less, 20 mass % or less, 10 mass % or less or 8 mass % or less, as a solid content relative to a total amount of the agent or composition.

(Ginkgo Leaf Extract)

The ginkgo leaf extract is an extract obtained from ginkgo (Ginkgo biloba L.) leaves. The ginkgo leaf extract itself has cerebral blood flow improving and anti-inflammatory effects and the like. The extraction solvent may be, for example, an alcohol such as ethanol, an organic solvent such as acetone, water or a mixed solution thereof. The extraction solvent is preferably water. Ginkgo leaves as an extraction raw material may be fresh, dried, or powdered. The ginkgo leaf extract may be a product obtained by performing additional treatments such as purification, concentration, and drying on the extract obtained from the ginkgo leaves. The ginkgo leaf extract may be a product from which ginkgolic acid has been removed by resin column purification or the like.

In the agent or composition according to the present embodiment, the content ratio between the propolis and the ginkgo leaf extract may be, for example, 1:0.2 to 5, 1:0.3 to 2, 1:0.5 to 2, 1:1 to 2 or 1:1.3 to 1.9 as a solid content.

The agent or composition, and can be used as the solid content of the ginkgo leaf extract, for example, at a dose of 1 mg to 1,000 mg, preferably at a dose of 10 to 500 mg and more preferably at a dose of 20 to 200 mg per day for an adult with a body weight of 60 kg.

In addition, the content of the ginkgo leaf extract in the agent or composition may be, for example, 1 mass % or more, 3 mass % or more, 5 mass % or more or 7 mass % or more, and may be 30 mass % or less, 20 mass % or less, 15 mass % or less or 12 mass % or less as a solid content relative to a total amount of the agent or composition.

(Phosphatidylserine)

The phosphatidylserine may be a synthetic product or may be a plant- or animal-derived product. Phosphatidylserine can be obtained by an enzymatic reaction using, for example, plant-derived lecithin such as soybean lecithin as a raw material. The phosphatidylserine itself has memory improving and cognitive function improving effects and the like.

In the agent or composition according to the present embodiment, the ratio between the propolis and phosphatidylserine may be, for example, 1:1 to 5, and is preferably 1:1 to 3 and more preferably 1:1.5 to 2.5.

The agent or composition can be used as the solid content of the phosphatidylserine, for example, at a dose of 1 mg to 1,000 mg, preferably at a dose of 10 to 500 mg and more preferably at a dose of 50 to 300 mg per day for an adult with a body weight of 60 kg.

The content of the phosphatidylserine in the agent may be, for example, 1 mass % or more, 5 mass % or more, 7 mass % or more or 10 mass % or more, and may be 30 mass % or less, 20 mass % or less, 17 mass % or less or 15 mass % or less, as a solid content relative to a total amount of the agent or composition.

(Coffee Fruit Extract)

The coffee fruit extract is a product obtained by extraction from coffee fruit. The coffee fruit is the fruit of the coffee tree, and may include exocarp, fruit pulp, mucilage, shells, stems and seeds. Arabica coffee seeds are preferably used. The extraction solvent may be, for example, an alcohol such as ethanol or methanol, an organic solvent such as acetone, water or a mixed solution thereof. The coffee fruit extract may be a product obtained by performing additional treatments such as purification, concentration and drying on the extract obtained from the coffee fruit.

The coffee fruit extract itself has neuroprotective effects and the like. When the coffee fruit extract is taken, the concentration of proteins in blood called a BDNF increases. The BDNF is a component necessary for promoting the production, growth and regeneration of nerve cells, and is also called brain nutrition.

In the agent or composition according to the present embodiment, the ratio between the propolis and the coffee fruit extract may be, for example, 1:0.5 to 3, preferably 1:0.8 to 2 and more preferably 1:1 to 1.7.

The agent or composition can be used as the solid content of the coffee fruit extract, for example, at a dose of 1 mg to 1,000 mg, preferably at a dose of 10 to 500 mg and more preferably at a dose of 20 to 200 mg per day for an adult with a body weight of 60 kg.

The content of the coffee fruit extract in the agent or composition may be, for example, 1 mass % or more, 3 mass % or more, 5 mass % or more or 7 mass % or more, and may be 30 mass % or less, 20 mass % or less, 15 mass % or less, 12 mass % or less or 10 mass % or less as a solid content relative to a total amount of the agent.

(Curcumine)

Curcumine is a pigment component contained in plants such as turmeric (Curcuma longa). Curcumine itself has antioxidation, anti-inflammatory, amyloid β inhibitory and intestinal barrier effects and the like. For example, curcumine can be extracted from turmeric rhizomes by a known method. For example, the turmeric extract may be a product obtained by extraction from turmeric rhizomes using water, hot water, an organic solvent such as ethanol or a mixed solution thereof as an extraction solvent. The agent or composition according to the present embodiment may contain, as a curcumine source, for example, turmeric root or an extract thereof. The agent or composition may contain turmeric-derived components other than curcumine. The curcumine may be a component of which absorbability has been enhanced by a known method such as micronization.

In the agent or composition according to the present embodiment, the ratio between the propolis and the curcumine may be, for example, 1:2 to 5, preferably 1:2.5 to 4 and more preferably 1:2.7 to 3.7.

The agent or composition can be used as the solid content of curcumine, for example, at a dose of 1 mg to 1,000 mg, preferably at a dose of 10 to 500 mg and more preferably at a dose of 100 to 350 mg per day for an adult with a body weight of 60 kg.

The content of the curcumine in the agent or composition may be, for example, 5 mass % or more, 10 mass % or more, 15 mass % or more or 17 mass % or more, and may be 40 mass % or less, 30 mass % or less, 25 mass % or less or 23 mass % or less as a solid content relative to a total amount of the agent or composition.

(Gotu Kola Extract)

The gotu kola extract can be obtained from extraction from gotu kola (Centella asiatica umbelliferae). The gotu kola extract itself has cognitive function improving and neurological disorder improving effects. Gotu kola is also called Centella asiatica. The extraction sites of gotu kola may be flowers, spikes, pericarps, fruits, stems, leaves, branches, foliage, trunks, bark, rhizomes, root bark, roots, seeds or whole plants. The extraction sites of gotu kola are preferably leaves, stems or whole plants. The extraction solvent may be, for example, water or an alcohol such as ethanol. The gotu kola extract may be a product obtained by performing additional treatments such as purification and drying on the extract obtained from a plant.

In the agent or composition according to the present embodiment, the ratio between the propolis and the gotu kola extract may be, for example, 1:1 to 6, and is preferably 1:2 to 5 and more preferably 1:2.5 to 4.

The agent or composition can be used as the solid content of the gotu kola extract, for example, at a dose of 1 mg to 1,000 mg, preferably at a dose of 10 to 500 mg and more preferably at a dose of 100 to 350 mg per day for an adult with a body weight of 60 kg.

The content of the gotu kola extract in the agent or composition may be, for example, 1 mass % or more, 5 mass % or more, 10 mass % or more, 15 mass % or more or 18 mass % or more, and may be 40 mass % or less, 30 mass % or less, 25 mass % or less or 23 mass % or less as a solid content relative to a total amount of the agent or composition.

The agent or composition particularly preferably contains at least a combination of propolis and a ginkgo leaf extract.

The agent or composition may not contain at least one of DHA and EPA or may contain neither DHA nor EPA. In particular, when the agent contains propolis and a ginkgo leaf extract, since these components are contained as active ingredients, a sufficiently strong effect can be obtained even if DHA and EPA are not contained. The agent or composition may or may not contain GABA.

The agent or composition can be used as a drug, quasi-drug or foodstuff itself, and can also be used as a component in a drug, quasi-drug or foodstuff.

The cause of Alzheimer's type dementia has been thought to be accumulation of amyloid β. However, in recent years, it has become clear that amyloid β is produced as a protective reaction against a plurality of threats such as inflammation, malnutrition and the presence of toxins in the brain. Therefore, it is thought that, in order to improve cognitive functions, eliminating the cause of its production is more effective than removing amyloid β itself.

Inflammation in the brain is said to be caused by, for example, excessive intake of trans fatty acids, gluten or casein, excessive intake of sugar and the like, and is also caused by obesity, deterioration of the oral environment or invasion of pathogens. Malnutrition of the brain is thought to be caused by lack of nutrients such as hormones, vitamins and minerals required for brain nerve cells, lack of exercise and the like. It is said that, when cranial nerves are undernourished, the brain atrophies. Toxins are caused by toxic metals, mold-derived toxins or the like. In addition, dementia such as Alzheimer's is also caused by glycose toxicity due to hyperglycemia as a mixed type of inflammatory and atrophy. In addition, damage caused by hemorrhage in the brain due to arteriosclerosis or the like may cause dementia. That is, there are five types of dementia such as Alzheimer's: inflammatory, glycotoxic, atrophic, toxic and vascular.

The agent or composition according to the present embodiment not only contains components which independently exhibit various effects such as an anti-inflammatory effect, antioxidation, antisaccharification, blood sugar level improvement, insulin resistance improvement, brain neuro protection, cranial nerve regeneration promotion, liver protection, protection of brain cells from endotoxins and cerebral blood flow improvement but also contains components in combination with propolis, and thus at least antioxidation, antisaccharification, the anti-inflammatory effect, neurite outgrowth promoting effects and the like are exhibited more strongly than a total of effects (additive effect) of each component contained alone. Therefore, the agent or composition according to the present embodiment exhibits a stronger cognitive function improving effect as a whole. In addition, the agent or composition according to the present embodiment also has liver function improving and sleep quality improving effects based on the anti-inflammatory effect. In addition, as described above, the agent or composition according to the present embodiment can be used as a cognitive function decline preventing agent because it leads to prevention of production and accumulation of amyloid β.

In order to exhibit strong effects for all of the five inflammatory, glycotoxic, atrophic, toxic and vascular types, it is particularly preferable that the agent according to the present embodiment contain all of the propolis, the ginkgo leaf extract, phosphatidylserine, the coffee fruit extract, the gotu kola extract and curcumine. Since any of the above components contained in the agent or composition according to the present embodiment can be used as a foodstuff, it has an advantage of being easily taken on a daily basis.

The agent according to the present embodiment can also be used as an agent for improving or preventing dementia such as Alzheimer's type dementia and an agent for improving or preventing mild cognitive impairment.

Subjects to whom the antioxidant agent, the antisaccharification agent, the neurite outgrowth promoting agent and the cognitive function improving agent according to the present embodiment are administered may be healthy people, or may be dementia patients such as Alzheimer's type dementia patients, people having mild cognitive impairment, which is a pre-stage of dementia, people at risk of cognitive function decline, people who are aware of their forgetfulness, people to whom others have brought up their forgetfulness, people having sleeping disorders and people who need to improve liver functions. The subjects may be, for example, people aged 45 years or older or may be elderly people, for example, people aged 60 years or older, 65 years or older, 70 years or older or 75 years or older.

The antioxidant agent, the antisaccharification agent, the neurite outgrowth promoting agent and the cognitive function improving agent according to the present embodiment are thought to be particularly effective in people with a low plasma brain-derived neurotrophic factor (BDNF) concentration and/or a low BDNF concentration in the brain. The plasma BDNF concentration and the BDNF concentration in the brain correlate with each other. Subjects to whom the antioxidant agent, the antisaccharification agent, the neurite outgrowth promoting agent and the cognitive function improving agent according to the present embodiment are administered may be people having a plasma BDNF concentration of, for example, 120 ng/ml or less, 110 ng/ml or less, 100 ng/ml or less, 90 ng/ml or less, 85 ng/ml or less, 79 ng/ml or less or 78 ng/ml or less. The plasma BDNF concentration of administration subjects may be 10 ng/ml or more, 30 ng/ml or more or 50 ng/ml or more. In addition, administration subjects may be people whose plasma BDNF concentration is equal to or less than the median value, equal to or less than the average value or less than the first quartile of the plasma BDNF concentration in a plurality of subjects.

The agent or composition according to the present embodiment may further contain other components. Examples of other components include pharmaceutically acceptable components (for example, excipients, binding materials, lubricants, disintegrators, emulsifiers, surfactants, bases, dissolution adjuvants and suspending agents) and components acceptable as foodstuffs (for example, minerals, vitamins, flavonoids, quinones, polyphenols, amino acids, nucleic acids, essential fatty acids, refreshing agents, binders, sweeteners, disintegrators, lubricants, colorants, fragrances, stabilizers, preservatives, sustained release adjusting agents, surfactants, solubilizers and wetting agents).

The agent or composition according to the present embodiment may be in any form such as a solid, a liquid or a paste, and may be a dosage form of tablets (including uncoated tablets, sugar-coated tablets, effervescent tablets, film-coated tablets, chewable tablets, troches and the like), capsules, pills, powder agents (powders), fine granules, granules, liquids, suspensions, emulsions, syrups, pastes and injection agents (including a form that is prepared as a liquid by being blended with distilled water or an infusion solution such as an amino acid infusion solution or an electrolyte infusion solution when used). These various formulations can be prepared by, for example, mixing the agent or composition obtained by the above method, and as necessary, other components, and molding it into the above dosage form.

When used as a foodstuff composition or one component of a foodstuff composition, the foodstuff composition is preferably a composition in which a foodstuff tertiary function, that is, a physical condition adjustment function, is enhanced. Examples of products in which a foodstuff tertiary function is enhanced include health foodstuffs, foodstuffs with function claims, nutritionally functional foodstuffs, nutritional supplement foodstuffs, supplements and specified health foodstuffs.

The agent or composition according to the present embodiment is preferably taken in the body. The administration mode may be oral administration or parenteral administration. The agent or composition according to the present embodiment may be administered once a day or may be administered separately in a plurality of doses such as twice a day or three times a day.

### EXAMPLES

Hereinafter, the present invention will be described in detail with reference to examples. However, the present invention is not limited to the following examples.

### Test Example 1: Evaluation of Neurite Outgrowth Activity

It has been reported that a nerve growth factor (NGF) is targeted and transported to cholinergic neurons to prevent cell death, and the NGF stimulates synaptic cholinergic receptors to promote cognitive improvement. Immature cells have a morphology close to a spherical shape in many cases and tend to have characteristic structures in the process of differentiating and imparting functionality. In the case of nerve cells, they form innumerable protrusions during final differentiation and become neurons. In this test, PC12 was primed in the direction of nerve differentiation by allowing NGF to act at a low concentration (10 ng/ml) in advance, and here, the sample was added, and thus the effect of the sample on an ability to induce neural differentiation was analyzed.

The following samples were used for evaluation.

Propolis (propolis ethanol extract, API propolis, EEP-B55A): propolis that was dissolved in ethanol for molecular biology at a concentration of 10 mg/mL, passed through a 0.2 μm filter, and then stored at −80° C. was used.

Ginkgo leaf extract (water extract, Tokiwa Phytochemical Co., Ltd.): a ginkgo leaf extract that was dissolved in dimethyl sulfoxide (DMSO) at 100 mg/mL, passed through a 0.2 μm filter, and then stored at −80° C. was used.

Nerve growth factor-β (derived from rats)

The following cells and mediums were used.

Cells: PC12 (JCRB0733, 06082017)

Medium: Completely Culture Medium (CCM)
RPMI 1640
10% horse serum
5% fetal bovine serum
1% penicillin-streptomycin (Nacalai Tesque)

Differentiation Medium (DM):
RPMI 1640
2% horse serum
1% fetal bovine serum
1% penicillin-streptomycin
10 ng/mL NGF-β

Culture conditions: 37° C., 5% CO₂

The CCM was used as the medium for subculturing PC12 cells. The subculture was performed when the cell density reached 70% confluency. Before use in the test, cell subculture was performed three times or more to provide a period for cell stabilization. During inducing of neurodifferentiation, 0.8×10⁴ cells diluted in 2 mL of CCM were seeded in a 6-well plate and cultured overnight. The medium was replaced with the DM, and culture was then performed for 48 hours. 48 hours after replacement with the DM, the medium was replaced with a DM medium in which addition conditions were changed for each well, and time-lapse observation was performed using a fluorescence microscope (BZ-X800 commercially available from Keyence Corporation). For addition conditions, propolis was added alone (25 μg/mL), the ginkgo leaf extract was added alone (10 μg/mL), and propolis (25 μg/mL) and the ginkgo leaf extract (10 μg/mL) were added together.

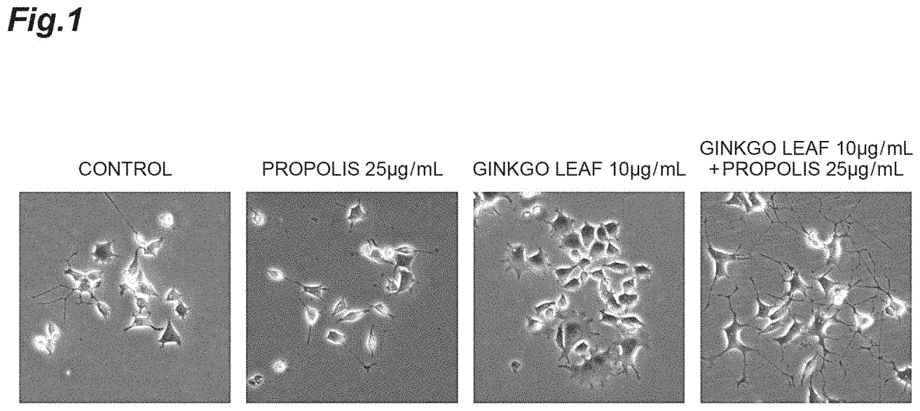

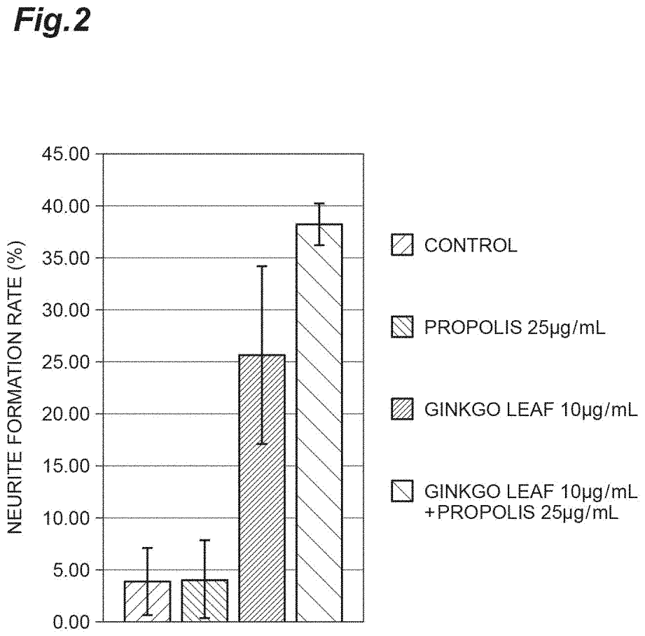

During culture for 17 hours, three pictures were taken for each well once every 15 minutes. The total number of cells within a field of view and the number of cells whose neurites were longer than the perikaryons were counted. The number of protrusions per cell was not considered, and a cell having at least one protrusion longer than the perikaryon was defined as a cell with an elongated protrusion. After the number of cells in each well was added, the numerical values in each condition were compared and analyzed. The number of cells with elongated protrusions with respect to the total number of cells observed after 17 hours was evaluated as the neurite formation rate (%). The results are shown in FIG. 1 and FIG. 2.

FIG. 1 shows phase contrast microscopic images of PC12 cells after culturing for 17 hours and shows cells from left to right: control (DMSO), propolis added alone, the ginkgo leaf extract added alone, propolis and the ginkgo leaf extract added together. FIG. 2 is a graph showing the neurite formation rate in the conditions. The error bar indicates mean±SD(n=3). It was shown that, compared to the case of the control and the cases in which propolis was added alone, and the ginkgo leaf extract was added alone, when propolis and the ginkgo leaf extract were added together, elongation of the protrusions of nerve cells was promoted.

### Test Example 2: Evaluation of Antioxidance

An antioxidance evaluation test was performed using menadione as an oxidative stress inducer. It has been reported that the treatment of menadione in cultured cells causes active oxygen (ROS) to be produced in the cells and induces cell death due to oxidative stress. Therefore, menadione was used to evaluate a material having an oxidative stress protective effect on cells.

Normal human keratinocytes were seeded at 3×10⁴ cells/cm² in a 24-well plate to which 500 μL of a keratinocyte growth medium was added in advance. After culturing for 24 hours, the medium was replaced with a medium in which menadione was added as an oxidative stress inducer to reach 0 or 100 μM. In addition, at the same time, 10 or 50 μg/ml of propolis, 17 or 86 μg/ml of the ginkgo leaf extract, or a combination of 10 or 50 μg/ml of the ginkgo leaf extract and 17 or 86 μg/ml of propolis at a concentration ratio of 1:1.7 was added in the medium to which 100 μM of menadione was added.

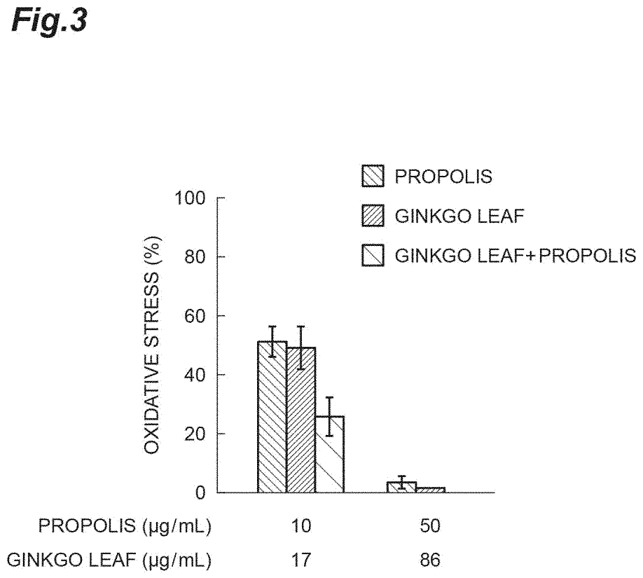

The oxidative stress level of cells 1 hour after a test substance was administered was evaluated using CellROX green, which is an oxidative stress detection reagent. In addition, the number of cells was evaluated according to the number of nuclei found through Hoechest 33342 staining. The oxidative stress level was calculated as the fluorescence intensity per cell obtained by dividing the fluorescence intensity of CellROXgreen by the number of nuclei, and the oxidative stress level of menadione alone was set to 100%. The results are shown in FIG. 3.

FIG. 3 is a graph showing the oxidative stress level at each concentration in the cases in which propolis was added alone, the ginkgo leaf extract was added alone, and the ginkgo leaf extract and propolis were added together. The error bar indicates mean±SD. In the cases in which propolis was added alone and the ginkgo leaf extract was added alone, the oxidative stress level was reduced in a concentration-dependent manner. When propolis and the ginkgo leaf extract were added together, a higher antioxidation activity was shown than when they were added alone. Based on the above results, it was shown that, when a combination of propolis and the ginkgo leaf extract was used, the oxidative stress protection effect was improved.

### Test Example 3: ORAC Test

The antioxidation capacity of the combination of propolis and each sample was verified by an oxygen radical absorption capacity (ORAC) test. The following samples were used.

Propolis: ethanol extract powder (Brazilian green propolis, containing arginine as an excipient)

Phosphatidylserine: synthetic product

Coffee fruit extract: water/ethanol extract powder

Gotu kola extract: water extract powder (containing less than 10% of maltodextrin as an excipient)

Each sample was dissolved in a 50% ethanol aqueous solution. As shown below, a sample that was not dissolved in an ethanol aqueous solution was suspended, subjected to shaking extraction at 100 rpm for 10 minutes and ultrasonic extraction for 10 minutes or subjected to only ultrasonic extraction for 10 minutes, and then centrifuged at 3,000 rpm for 10 minutes to obtain a supernatant.

Phosphatidylserine: after shaking extraction and ultrasonic extraction, centrifuge
Propolis+phosphatidylserine: ultrasonic extraction only (no centrifuge)
Gotu kola extract, propolis+gotu kola extract: after ultrasonic extraction, centrifuge
Sample other than the above samples: dissolution only (no extraction process/centrifuge)

The obtained test solution was diluted with a 75 mM phosphate buffer and put into a 96-well plate, and a fluorescein solution was added thereto. When the 96-well plate was heated to 37° C., AAPH (2,2′-azobis(2-amidinopropane)dihydrochloride) was added, and the fluorescence intensity decay time was measured with a microplate reader every 5 minutes for 1.5 hours. A standard curve was created using Trolox as a standard reagent, and the antioxidation power of each sample was calculated as a Trolox equivalent (μmol TE/g). The results are shown in Table 1.

TABLE 1

| | Single raw | Combination/content ratio | | |
|---|---|---|---|---|
| | material | Propolis × | Propolis × | Propolis × |
| Raw | ORAC | Phosphatidyl- | Coffee fruit | Gotu kola |
| material | (μmol TE/g) | serine | extract | extract |
| Propolis | 3900 | 31.85% | 39.71% | 20.86% |
| Phosphatidyl- | 12 | 68.15% | | |
| serine | | | | |
| Coffee fruit | 10000 | | 60.29% | |
| extract | | | | |
| Gotu kola | 44.0 | | | 79.14% |
| extract | | | | |
| ORAC | Expected | 1250 | 7578 | 1161 |
| (μmol TE/g) | value | | | |
| | Measured | 1600 | 8000 | 1400 |
| | value | | | |
| | Rate of | 128.0% | 105.6% | 120.6% |
| | increase | | | |

In Table 1, the expected value is a sum of ORAC values of respective components alone in each combination. The rate of increase is a value obtained by multiplying a result obtained by dividing the measured value by the expected value by 100. It was shown that, when phosphatidylserine, the coffee fruit extract, and the gotu kola extract were combined with propolis, the antioxidation capacity synergistically increased.

### Test Example 4: Measurement of AGEs

The antisaccharification capacity of the combination of propolis and each sample was verified by an advanced glycation end products (AGEs) measurement test. Regarding the samples, propolis, phosphatidylserine, a coffee fruit extract, curcumine and a ginkgo leaf extract were used. The same propolis, phosphatidylserine and coffee fruit extract as in Test Example 3 were used. Regarding the ginkgo leaf extract, a ginkgo leaf ethanol/water extract powder was used. Regarding the curcumine source, a turmeric extract (ethyl acetate extract powder, curcumine content of 95 mass %) was used.

Each sample was dissolved in 100% dimethyl sulfoxide. 12 of the obtained solution or ultrapure water, 40 μL of a 20 mg/mL human serum albumin solution, and a 0.4 mol/L glucose solution or 50 μL of ultrapure water were put together into a 1.5 mL tube. The liquid in the tube was mixed well using a vortex and then incubated at 60° C. for 40 hours. The solution after the reaction was dispensed into a 384-well plate, excitation light at 370 nm was emitted, the fluorescence at 440 nm was measured, and the amount of AGEs produced was calculated. The AGEs production inhibition rate was calculated by the following formula. The results are shown in Table 2.

AGEs production inhibition rate (%)={1−(A−B)/(C−D)}×100

A: amount produced in a mixed solution of the sample solution, the albumin solution and the glucose solution
B: amount produced in a mixed solution of the sample solution and the albumin solution
C: amount produced in a mixed solution of the albumin solution and the glucose solution
D: amount produced in the albumin solution

TABLE 2

| | Single raw | | | | |
|---|---|---|---|---|---|
| | material AGEs | Combination/content ratio | | | |
| | production | Propolis × | Propolis × | | Propolis × |
| Raw | inhibition | Phosphatidyl- | Coffee fruit | Propolis × | Ginkgo leaf |
| material | rate (%) | serine | extract | Curcumine | extract |
| Propolis | 4.27 | 31.75% | 40% | 22.73% | 37.04% |
| Phosphatidyl- | −12.0 | 68.25% | | | |
| serine | | | | | |
| Coffee fruit | 11.9 | | 60% | | |
| extract | | | | | |
| Turmeric | 49.1 | | | 77.27% | |
| extract | | | | | |
| Ginkgo leaf | −4.86 | | | | 62.96% |
| extract | | | | | |
| AGEs | Expected | −7.7 | 16.2 | 55.3 | −0.6 |
| production | value | | | | |
| inhibition | Measured | 8.8 | 22.7 | 71.8 | 14.9 |
| rate (%) | value | | | | |
| | Amount | 16.5 | 6.5 | 16.5 | 15.5 |
| | increased | | | | |

In Table 2, the expected value of the AGEs production inhibition rate in each combination is a total value of the production inhibition rates shown by respective components alone used in the combination. The amount of increase is a value obtained by subtracting the expected value from the measured value. When propolis was combined with phosphatidylserine, the coffee fruit extract, curcumine or the ginkgo leaf extract, the AGEs production inhibition rate was significantly higher than when they were used alone. It was confirmed that, when propolis was combined with phosphatidylserine, the coffee fruit extract, curcumine or the ginkgo leaf extract, a stronger antisaccharification effect was obtained compared to when components were used alone.

[Test Example 5: Evaluation of Nrf2 pathway activation] Nrf2 is a transcription factor that is activated in response to oxidative stress, and genes involved in antioxidation, and detoxification metabolism are present downstream therefrom. It has been reported that the Nrf2 pathway affects dementia at the animal level, and activation of Nrf2 is expected to improve cognitive functions. In this test, PC12/ARE reporter cells in which ARE, which is a transcription response sequence downstream of Nrf2, was incorporated into a reporter vector were used for the test, and a synergistic effect of Nrf2 pathway activation was examined.

<Cell Culture>

PC12/ARE reporter cells (hereinafter referred to as PC12/ARE) distributed by Kobe Pharmaceutical University were used. The cells were PC12 cells in which a promoter region containing the ARE sequence of the rat-derived NADPH: quinone oxidoreductase 1 (NQO1) gene, which is a downstream gene of Nrf2, was incorporated upstream from the luciferase gene, and stably expressed.

Basic medium: which was prepared by adding each factor to a RPMI-1640 medium (Nacali, Code: 30264-85) so that 10% fetal bovine serum (FBS, BioWest), 5% horse serum (HS, Gibco), 1× penicillin/streptomycin (Nacali, Code: 09367-34), and 200 μML-Glutamine (Nacali, Code: 16948-04) were obtained.

Maintenance medium: which was prepared by adding Hygromycin B (Wako, Cat: 085-06153) to the basic medium so that the concentration was 300 μg/mL for reporter cell line selection.

PC12/ARE was maintained and subcultured according to the PC12/ARE culture protocol. The medium was replaced once every 2 to 3 days.

<Reporter Assay>

PC12/ARE was seeded in a 384-well plate (White plate) so that 1×10⁴ cells/20 μL/well. The basic medium was used for culturing. After culturing overnight, a 5 μL sample was added, and 24 hours later, 25 μL of ONE-Glo (registered trademark) Luciferase Assay Reagent (Promega, Cat: E6120) was added, and after incubation for 3 minutes, the reporter activity was measured by Envision (PerkinElmer).

<Sample Preparation>

Propolis, curcumine and the ginkgo leaf extract were suspended in DMSO at 50 mg/ml, stirred for 1 hour, and then centrifuged to obtain a supernatant. The same propolis, curcumine and ginkgo leaf extract used in Test Example 3 or 4 were used. The supernatant was filtered through a 0.22 μm filter and then dispensed, and stored at −30° C. Each sample was dissolved during the test and diluted with DMSO and the basic medium.

<ARE Reporter Activity>

Each of propolis at a concentration of 4.5 μg/ml, curcumine at a concentration of 13.8 μg/ml, and the ginkgo leaf extract at a concentration of 6.6 μg/ml was added alone to or combinations shown in Table 3 were added to PC12/ARE cells, and the reporter activity after 24 hours was measured. As a result, in the combination of propolis and curcumine, and the combination of propolis, curcumine and the ginkgo leaf extract, a synergistic effect was observed compared to when respective components were added alone.

TABLE 3

| | ARE reporter |
|---|---|
| | activity (RLU) |
| Propolis | 716 |
| Curcumine | 5748 |
| Ginkgo leaf | 1419 |
| Propolis + Curcumine | 11665 |
| Propolis + Curcumine + | 24336 |
| Ginkgo leaf | |

### Test Example 6: Human Biological Test

A complex supplement containing a propolis extract, a ginkgo leaf extract, phosphatidylserine, curcumine, a coffee fruit extract, and a gotu kola extract was administered to humans to verify the effect.

<Subjects>

90 people who met all of the following selection criteria and did not meet any of the following exclusion criteria were test subjects.

1) Selection criteria
Men and women whose ages were 40 or more and less than 80 when consent was obtained
People with a Mini-Mental State Examination (MMSE) of 24 points or more and 29 points or less
People who were aware of their forgetfulness or people to which others had brought up their forgetfulness
   2) Exclusion criteria
People who have been diagnosed with dementia by doctors or people with diseases that may affect cognitive functions
People who are taking dementia therapeutic drugs or people who have taken dementia therapeutic drugs
People who are taking regularly drugs that may affect cognition (first-generation antihistamines, benzodiazepines, sedatives, opiates, stabilizers, antidepressants, cholinergic agents, anticholinergic agents, and prescription anti-inflammatory agents)
People with a history of present illness or a past medical history of mental disorders (including depressive symptoms) or cerebrovascular diseases
People who taking supplement-health foodstuffs (including foodstuffs with function claims) that may affect cognitive functions
People found through the subject background questionnaire to have had extremely irregular lifestyle habits related to eating, sleeping, etc.
People with a geriatric depression scale (Geriatric Depression Scale-Short Version-Japanese: GDS-S-J) of 6 points or more
People with a past medical history or a history of present illness of alcohol dependence
People who consume a large amount of alcohol on a daily basis (people who drink 14 or more 350 mL servings of beer, and 180 mL servings of wine a week)
People with a history of present illness or a past medical history of severe illnesses such as diabetes, liver disease, kidney disease, and heart disease.
People with a past medical history or a history of present illness of drug dependence or food allergies
People with color-vision deficiency and people who have difficulty hearing even at short distances
People with problems with the function of both hands due to injury, surgery or the like

<Test Foodstuffs>

The amount of the complex supplement administered was three capsules (hard capsules) per day. The complex supplement contained, in three capsules, propolis (4.25 mg as artepillin C), 175 mg of curcumine, 100 mg of soybean-derived phosphatidylserine, the ginkgo leaf extract (28.8 mg as ginkgo leaf-derived flavonoid glycoside, and 7.2 mg as ginkgo leaf-derived terpene lactone), the gotu kola extract (225 mg), and the coffee fruit extract (100 mg). Respective components were the same as those used in Test Example 3 or 4. The complex supplement further contained fine silicon dioxide as an additive. The placebo was made by replacing the above components with starch, and the complex supplement and the placebo were made similar in appearance to each other so that they could not be distinguished between foodstuffs. The component compositions (daily intake, per three capsules) of the complex supplement and the placebo are shown in Table 4.

TABLE 4

| | Placebo | Complex supplement |
|---|---|---|
| Calorie (kcal) | 4.0 | 5.2 |
| Protein (g) | 0.2 | 0.3 |
| Lipid (g) | 0.3 | 0.2 |
| Carbohydrate (g) | 0.9 | 0.5 |
| Salt equivalent (mg) | 3.1 | 11.0 |

<Test Method and Schedule>

This test was deliberated and approved by the Nihonbashi Cardiovascular Department Clinic Ethics Committee and compiled with the Declaration of Helsinki (amended by VMA Fortaleza General Assembly, 2013), and ethical guidelines for medical research involving human subjects. The test protocol was registered in the University Hospital Medical Network Clinical Trial System.

The test design was a placebo-controlled, randomized, parallel-group, double-blind, and comparative human clinical test, the purpose, method and the like of this test were fully explained to the subjects, and then voluntary written consent was obtained. The subjects were divided into two groups with even distribution of age, sex, body-mass index (BMI), and MMSE.

Pre-intake tests (height, body weight, blood pressure, pulse, and MMSE, GDS-S-J, Mild Cognitive Impairment (MCI) screen, cognitrax, blood test, various Visual Analog Scales (VAS), MOS 36-Item Short-Form Health Survey (SF-36) conducted by clinical psychologists) were conducted in October-November 2019. From mid-November 2019 to late February 2020, three capsules of the placebo or the complex supplement were taken daily together with water at room temperature for 12 weeks. In addition, 12 weeks after administration, height, body weight, blood pressure, pulse, MCI screen, cognitrax, blood tests, various VAS, and SF-36 were examined. In addition, during the test period, whether test foodstuffs were taken, whether physical conditions had changed, whether living conditions had changed, an exercise status, an intake status of drugs and health foodstuffs, an interpersonal relationship status, and a sleeping status were recorded daily in the diary.

<Test Items>

### (MCI Screen)

The term mild cognitive impairment (MCI) is used to indicate a precursor state of dementia. Since early determination of dementia is important with the development of new treatment methods, the diagnosis of MCI has been focused on. The MCI screen is a cognitive function test developed by Shankle et al., and for comprehensively evaluating memory and attentiveness. Among MCI screen tests, MPI scores and immediate memory task scores were measured. The MPI score was used to mainly comprehensively evaluate scores of the immediate memory task and the delayed memory task.

(Cognitrax)

The cognitrax is a general cognitive test based on CNS Vital Sign. In this test, a verbal memory test (VBM), a visual memory test (VIM), a finger tapping test (FTT), an SDC test (SDC), a Stroop test (ST), a shifting attentiveness test (SAT), a continuous performance test (CPT), and a 4-part continuous performance test (FPCPT) were performed, and the evaluation was performed using a standardized score (average 100), which is a conversion value compared to the same age group. Based on these tests, the incorrect ST responses were evaluated.

(Blood Test)

The subject's blood was collected, and a tumor necrosis factor (TNF-α, serum), BDNF (plasma), and the amount of AST as a liver function indicator were measured. TNF-α is known as a cytokine deeply involved in the biophylactic mechanism through inflammation. AST is released into blood when hepatocytes are damaged, and inflammation is thought to be one of the causes. TNF-α and AST were evaluated by LSI Medience Corporation, and BDNF was evaluated by Japan Institute for the Control of Aging, Nikken Seil Co., Ltd.

(Subjective Symptoms (VAS))

VAS is a test for detecting subjective symptoms. The sleep quality was evaluated by asking “How is the quality of your sleep?” A straight line was shown on the screen, the left end (0) of the straight line was set as “very bad,” the right end (100) of the straight line was set as “very good,” and the state of the subject at that time was marked on the straight line.

(SF-36)

Among SF-36 measures, which are evaluation scales for quality of life (QOL), the social functioning scale (the extent to which physical or psychological problems have interfered with social activity in the past month) was evaluated. The score was scored (norm-based scoring: NBS) based on the national standard value and calculated as a standard score (average 50).

<Statistical Processing>

The measured values were shown as mean±standard deviation. Intra-group comparison between before administration and 12 weeks after administration was performed by paired t-test, and inter-group comparison of the complex supplement group with respect to the placebo group was performed by Student's t-test. In addition, the amount of change (A) after 12 weeks based on before administration was also compared between groups in the same manner. All tests were two-sided tests. SPSS Ver. 25 (commercially available from IBM) was used as statistical analysis software.

(Analysis Subjects)

A total of 90 people (45 people in each group) were participated as subjects in the test. Among them, a total of 7 people including two persons whose liver function indicators AST, ALT and γ-GTP, which have been reported to be associated with cognitive functions, had decreased from an abnormal value (before administration) to a half value or less (12 weeks after administration) (one person in the placebo group and one person in the complex supplement group), one person whose fluctuation of blood pressure which has been reported to be associated with cognitive functions, before and after administration was an outlier (<mean−3SD) (complex supplement group), one person who stopped the blood pressure therapeutic drug they were taking when they started eating the test meal during the test period (placebo group), two persons whose fluctuations of hsCRP, which is one inflammatory marker which has been reported to be associated with cognitive functions before and after administration, were outliers (>mean+3SD) (one person in the placebo group and one person in the complex supplement group), and one person whose fluctuation of body weight before and after administration was an outlier (<mean−3SD) were excluded as analysis subjects because it was determined that it was highly likely that they would not be able to maintain regular lifestyle habits, which could adversely affect the accuracy of effect verification on the cognitive functions.

One person in which the MCI screen test time after administration was much shorter than that before administration and was an outlier (<mean−3SD) was excluded as an analysis subject because it was determined that there was a high possibility of the evaluation not being clearly and appropriately performed in the cognitive function test 12 weeks after administration. Therefore, the number of cases to be analyzed was 82 people. In addition, for one person who gave up the 4-part continuous performance test for the cognitrax after administration during the test (complex supplement group), the item related to the result of the 4-part continuous performance test after administration was set as a missing value. Table 5 shows the background of the subjects to be analyzed. No significant difference was observed between groups in age, sex, BMI or MMSE.

TABLE 5

| | Placebo group | Complex supplement | |
|---|---|---|---|
| | (n = 42) | group (n = 40) | P value |
| Age (years) | 65.1 ± 7.3 | 65.3 ± 6.1 | 0.879 |
| Sex (male:female) | 20:22 | 21:19 | — |
| BMI (kg/m²) | 23.4 ± 2.5 | 23.5 ± 3.0 | 0.892 |
| MMSE | 27.6 ± 1.2 | 27.6 ± 1.2 | 0.872 |

<Results>

### (Cognitive Function Test)

The MCI screen results are shown in Table 6. Compared to the placebo group, in the complex supplement group, significant improvements were observed in the amounts of change (Δ) in the MPI score and the immediate memory task score 12 weeks after administration compared to before administration (P=0.047 and P=0.010).

TABLE 6

| | | Placebo group | Complex supplement |
|---|---|---|---|
| | | (n = 42) | group (n = 40) |
| MPI score | Before | 64.3 ± 7.0 | 61.7 ± 7.3 |
| | administration | | |
| | 12 weeks after | 63.8 ± 7.8 | 64.0 ± 8.6^{#} |
| | administration | | |
| | Δ | −0.5 ± 7.1 | 2.3 ± 5.5* |
| Immediate | Before | 20.1 ± 3.4 | 18.8 ± 3.7 |
| memory | administration | | |
| task score | 12 weeks after | 19.6 ± 3.2 | 20.3 ± 4.2^{##} |
| | administration | | |
| | Δ | −0.5 ± 3.7 | 1.5 ± 3.1* |

mean ± standard deviation
*P < 0.05 (vs. placebo group)
^{#}P < 0.05,
^{##}P < 0.01 (vs. before administration)

The cognitrax results are shown in Table 7.

Compared to the placebo group, in the complex supplement group, a significant improvement (P=0.023) was observed in the amount of change (Δ) in the incorrect ST responses, which is a component of total attentiveness and cognitive flexibility.

TABLE 7

| | | Placebo group | Complex supplement |
|---|---|---|---|
| | | (n = 42) | group (n = 40) |
| Incorrect ST | Before | 103.3 ± 6.9 | 103.1 ± 7.2 |
| response | administration | | |
| | 12 weeks after | 99.1 ± 14.2^{#} | 104.6 ± 6.7* |
| | administration | | |
| | Δ | −4.2 ± 13.2 | 1.5 ± 8.3* |

mean ± standard deviation
*P < 0.05 (vs. placebo group)
^{#}P < 0.05 (vs. before administration)

The blood test results are shown in Table 8. Compared to the placebo group, in the complex supplement group, significant improvements were observed in the amounts of change (Δ) of AST and TNF-α 12 weeks after administration compared to before administration (P=0.014 and P=0.026).

TABLE 8

| | | Placebo group | Complex supplement |
|---|---|---|---|
| | | (n = 42) | group (n = 40) |
| AST (U/L) | Before | 22.5 ± 5.8 | 24.1 ± 8.1 |
| | administration | | |
| | 12 weeks after | 23.3 ± 6.7 | 22.2 ± 5.2^{#} |
| | administration | | |
| | Δ | 0.8 ± 4.8 | −1.9 ± 4.8* |
| TNF-α | Before | 5.35 ± 1.92 | 6.21 ± 3.65 |
| (pg/mL) | administration | | |
| | 12 weeks after | 5.58 ± 2.25 | 5.50 ± 2.93^{#} |
| | administration | | |
| | Δ | 0.24 ± 2.03 | −0.72 ± 1.75* |

mean ± standard deviation
*P < 0.05 (vs. placebo group)
^{#}P < 0.05 (vs. before administration)

The subjective symptoms (VAS) results are shown in Table 9. Compared to the placebo group, in the complex supplement group, a significant improvement (P=0.016) was observed in the amount of change (Δ) of subjective symptoms of the sleep quality 12 weeks after administration compared to before administration.

TABLE 9

| | | Placebo group | Complex supplement |
|---|---|---|---|
| | | (n = 42) | group (n = 40) |
| Sleep | Before | 68.2 ± 19.7 | 59.4 ± 20.2* |
| quality | administration | | |
| | 12 weeks after | 68.3 ± 18.4 | 67.5 ± 18.4^{##} |
| | administration | | |
| | Δ | 0.0 ± 13.2 | 8.1 ± 16.3* |

mean ± standard deviation
*P < 0.05 (vs. placebo group)
^{##}P < 0.01 (vs. before administration)

The SF-36 results are shown in Table 10. Compared to the placebo group, in the complex supplement group, a significant improvement (P=0.030) was observed in the amount of change (Δ) in the social functioning scale (the extent to which physical or psychological problems have interfered with social activity in the past month) 12 weeks after administration compared to before administration.

TABLE 10

| | | Placebo group | Complex supplement |
|---|---|---|---|
| | | (n = 42) | group (n = 40) |
| Social | Before | 54.9 ± 4.6 | 50.9 ± 10.0* |
| functioning | administration | | |
| scale | 12 weeks after | 54.1 ± 6.8 | 53.3 ± 7.6^{##} |
| | administration | | |
| | Δ | −0.8 ± 7.6 | 2.4 ± 5.2* |

mean ± standard deviation
*P < 0.05 (vs. placebo group)
^{##}P < 0.01 (vs. before administration)

As a result of verification of the effect of complex supplement administration on the cognitive functions in this test, compared to the placebo group, in the complex supplement group, significant improvements were observed in the incorrect ST responses (cognitrax), the MPI score (MCI screen), the immediate memory task score (MCI screen), TNF-α, AST, subjective symptoms of the sleep quality, and the social functioning scale (SF-36).

The ST test is a test in which participants answer questions on the correspondence between the meaning of text and a color shown on a screen, and is known as a test capable of evaluating attentiveness and concentration that allow one to focus on target information when two different types of information, word information and color vision information, interfere with each other, and judgment ability that allows one to process information and make accurate judgments, and the like. In addition, since incorrect ST responses (wrong answers on the ST test) are items constituting total attentiveness and cognitive flexibility (ability to process in response to changes in instructions judgment ability), fewer incorrect ST responses is thought to indicate improvement in concentration (ability to maintain concentration), attentiveness (ability to maintain attentiveness and respond accurately), and judgment ability (ability to process information and make accurate judgements). In addition, since the MCI screen is a cognitive function test that requires verbal memory and attentiveness developed by Shankle et al., it is thought that improvement in MPI score, which is a total score, indicates improvement in verbal memory and attentiveness, and improvement in attentiveness matches the cognitrax result. Specific examples of improving attentiveness in daily life include “Driving a car safely for a long time” and “Not overlooking a red light.”

<Subgroup Analysis>

It is known that BDNF in the brain is lower in dementia patients than in healthy subjects, and the BDNF concentrations in blood and the brain are correlated. Therefore, the group in which the BDNF concentration was less than the first quartile was classified as a group with a high risk of cognitive function decline, and subgroup analysis was performed on the cognitrax test results. Here, no significant difference was observed between groups in background age, sex, BMI or MMSE (Table 11).

The analysis results are shown in Table 12. Compared to the placebo group, in the complex supplement group, in the amount of change (Δ) 12 weeks after administration compared to before administration, significant improvements were observed in the neurocognitive index (P=0.001), the cognitive flexibility (P=0.022), the executive function (P=0.026) and the SAT correct response (P=0.012), which is a component of cognitive flexibility.

TABLE 11

| | Placebo group | Complex supplement | |
|---|---|---|---|
| | (n = 10) | group (n = 10) | P value |
| Age (years) | 68.8 ± 5.0 | 66.8 ± 7.3 | 0.485 |
| Sex (male:female) | 5:5 | 4:6 | — |
| BMI (kg/m²) | 22.2 ± 2.5 | 23.5 ± 1.9 | 0.203 |
| MMSE | 27.8 ± 1.2 | 27.4 ± 1.4 | 0.511 |

TABLE 12

| | | Placebo group | Complex supplement |
|---|---|---|---|
| | | (n = 10) | group (n = 10) |
| Neurocognitive | Before | 107.2 ± 6.5 | 94.3 ± 12.7* |
| index | administration | | |
| | 12 weeks after | 104.7 ± 6.9 | 99.8 ± 8.3^{#} |
| | administration | | |
| | Δ | −2.5 ± 4.6 | 5.5 ± 7.4** |
| Cognitive | Before | 105.8 ± 11.3 | 89.2 ± 16.5* |
| flexibility | administration | | |
| | 12 weeks after | 106.0 ± 12.7 | 100.3 ± 12.9^{#} |
| | administration | | |
| | Δ | 0.2 ± 6.3 | 11.1 ± 12.3* |
| Executive | Before | 105.4 ± 11.6 | 88.6 ± 16.8* |
| function | administration | | |
| | 12 weeks after | 105.9 ± 13.2 | 99.6 ± 13.1^{#} |
| | administration | | |
| | Δ | 0.5 ± 6.1 | 11.0 ± 12.2* |
| Correct SAT | Before | 103.0 ± 12.7 | 85.5 ± 15.3* |
| response | administration | | |
| | 12 weeks after | 102.7 ± 14.3 | 96.0 ± 12.7^{#} |
| | administration | | |
| | Δ | −0.3 ± 6.2 | 10.5 ± 10.5* |

mean ± standard deviation
*P < 0.05,
**P < 0.01 (vs. placebo group)
^{#}P < 0.05 (vs. before administration)

As a result of subgroup analysis regarding cognitrax in the group in which the BDNF concentration was less than the first quartile, significant improvements were observed in the neurocognitive index, the cognitive flexibility, the executive function (ability to understand rules and concepts and make decisions), and the SAT correct response, which is a component of cognitive flexibility. The cognitive flexibility was calculated according to “correct SAT response-wrong SAT answer-incorrect ST response,” the executive function was calculated according to “correct SAT response-wrong SAT answer,” the SAT test is a test in which participants answer questions on the correspondence between a shape and a color of a figure shown on a screen, which is a very similar to the ST test, and thus improvements in cognitive flexibility and executive function are all thought to indicate improvements in attentiveness, concentration, and judgment ability.

In this test, in addition to improvement in cognitive functions, improvement in TNF-α was observed. Since it has been reported that people with acute or chronic systemic inflammation have a 2 to 4 times the rate of cognitive function decline of people without systemic inflammation, the complex supplement may contribute to improving sleep quality according to the anti-inflammatory effect. In addition, since it has been reported that the sleep quality and inflammation are related and it has been reported that an anti-TNF-α therapy contributes to improving sleep quality, the complex supplement is thought to contribute to improving sleep quality according to the anti-inflammatory effect. In addition, since it has been reported by meta-analysis that the risk of developing dementia is 1.51 times higher in cases of insomnia, improvement in subjective symptoms of sleep quality is also thought to contribute to improving cognitive functions.

In addition, if AST found to be improved in this test is released into blood when hepatocytes are damaged, inflammation is thought to be one of the causes. Therefore, it is thought that one mechanism for improving the liver function marker may be an anti-inflammatory effect. In addition, the social functioning scale, which is one of the items constituting SF-36 found to be improved in this test, is an index indicating the extent to which physical or psychological problems have interfered with social activity in the past month. Like phenomena such as “I can't immediately say what I want to say,” “I don't remember what I said,” “I can't remember what I was doing,” and “I am repeatedly buying foodstuffs that there are already many of in the refrigerator,” the cognitive function decline is thought to affect social life such as socializing. Therefore, the effectiveness for the cognitive function recognized this time is expected to lead to improvement in social functioning scale.

As described above, it was confirmed that, according to the combination of propolis and curcumine, the Nrf2 activity was synergistically improved. In addition, as shown in Test Example 1, it was confirmed that, according to the combination of propolis and ginkgo leaves, neurite outgrowth was synergistically promoted. Nrf2 is a transcription factor that is activated in response to oxidative stress, and it has been reported that the cognitive function was improved according to Nrf2 activation, and neurite outgrowth is thought to contribute to brain development. Therefore, respective components containing propolis are thought to synergistically exhibit antioxidation, anti-inflammatory and neurite outgrowth promoting effects, and thus exhibit an effect on maintenance and improvement of the cognitive functions. It was confirmed that the complex supplement improved not only the cognitive function such as verbal memory, attentiveness, concentration, and the judgment ability but also the sleep quality and the liver function conditions according to the antioxidation, antisaccharification, anti-inflammatory effects and the like.

1.-8. (canceled)

9. A composition comprising propolis, and at least one of a ginkgo leaf extract, a coffee fruit extract, a gotu kola extract, curcumine and phosphatidylserine.

10. (canceled)

11. A composition comprising propolis, a ginkgo leaf extract, a coffee fruit extract, a gotu kola extract, curcumine, and phosphatidylserine.

12. A method for improving cognitive function by using a capsule comprising the composition according to claim 9.

13. A method for improving cognitive function by using a capsule comprising the composition according to claim 11.
